(19) United States
(12) Patent Application Publication
(10) Pub. No.: US 2002/0022228 A1
(43) Pub. Date: Feb. 21, 2002

(54) Method and test kit for analyzing DNA repair

(76) Inventors: Peter Nehls, Kirchheim (DE); Karl Heinz Glusenkamp, Essen (DE)

Correspondence Address: DARBY & DARBY P.C., 805 Third Avenue, New York, NY, 10022, US

(21) Appl. No.: 09/848,116

(22) Filed: Apr. 30, 2001

Related U.S. Application Data
(63) Continuation of application No. PCT/EP99/08365, filed on Nov. 2, 1999.

(30) Foreign Application Priority Data
Nov. 3, 1998 (DE) 198 50 680.5

(51) Int. Cl. C12Q 1/68; C07H 21/04

(52) U.S. Cl. 435/6; 536/23.1

(57) ABSTRACT

A method for analyzing the repair of DNA modifications and base mispairings as well as apurinic and apyrimidinic sites by DNA repair enzymes, comprising the following steps: contacting (a) single- or double-stranded DNA molecules which were covalently coupled to a solid-phase matrix carrying primary or secondary amino groups by reaction with a reactive squaric acid derivative, and which have modifications and/or base mispairings and/or apurinic or apyrimidinic sites, with (b) a composition containing DNA repair enzymes; and determining the elimination of the DNA modifications and/or base mispairings and/or apurinic or apyrimidinic sites. The DNA molecules are covalently coupled to the solid state matrix via a primary or secondary amino group incorporated in the DNA molecule at the 5′-end or at the 3′-end of the DNA or in the 2′-position of at least one deoxyribosyl residue.

The present invention relates to a method and a test kit for analyzing the repair of DNA modifications and base mispairings as well as apurinic or apyrimidinic sites by DNA repair enzymes. It is thus possible, for instance, to determine the repair capacity of the repair enzymes and hence the repair capacity of cells or tissues from which compositions used in the method and containing repair enzymes were recovered. The repair capacity is relevant, for instance, in connection with processes which lead to the development of cancer, but is also important in various forms of cancer therapy.

A. Introduction

Cancer develops in several stages due to the gradual accumulation of mutations in the cancer-relevant genes (protooncogenes and tumor-suppressor genes) of a cell. For the development of mutations, especially carcinogenic factors play a role, which occur, for instance, in the environment, in food-stuffs, cosmetics, drugs and at the working place (UV-light, ionizing radiation, dusts, heavy metals and the multitude of chemical carcinogens), but may also be formed endogenously (e.g. nitrosamines, reactive nitrogen and oxygen compounds).

Despite their different physical and chemical properties, all carcinogens act according to the same principle. They react with the individual components of the DNA and thus change the structure and properties thereof.

However, structural changes of the DNA components also occur without the influence of carcinogens. Such structural changes are obtained by thermal hydrolysis of chemical bonds in the DNA. They include above all the replacement of the amino groups in the bases cytosine, adenine or guanine by hydroxyl groups (deamination) and the hydrolytic cleavage of the N-glycosidic bond between (in particular) the purine bases guanine and adenine and the deoxyribose part of the DNA (depurination).

Base mispairings can occur during the DNA replication, when too little or too much nucleotides and/or wrong nucleotides are incorporated in the newly synthesized DNA strands. The latter happens when the four DNA bases are present in their rare tautomeric form. For instance, cytosine in the rare tautomeric form forms a base pair with adenine and not with guanine, guanine in the rare tautomeric form forms a base pair with thymine and not with cytosine, etc.. If these or the other possible base mispairings are not repaired in time, transition mutations are obtained in the genomic DNA after another round of replications (exchange e.g. of C-G by T-A or of T-A by C-G). These transition mutations are then always passed on to the daughter cells. Structural modifications can produce all kinds of possible mutations (transitions, transversions, deletions, insertions, etc.). Type and distribution pattern of the mutations obtained in the genome are often characteristic for the carcinogen, which is responsible for the structural modifications obtained. The successive accumulation of mutations in the cancer-relevant genes (protooncogenes, tumor-suppressor genes) finally leads to the expression of the malignant phenotype of a cell.

Moreover, the influence of agents damaging the DNA may, however, directly result in the death of a cell concerned. This is important, for instance, in the therapy of tumor diseases by means of radiation or genotoxic chemotherapeutic agents.

For its protection, the cell has a number of effective defense mechanisms. These include on the one hand enzymes (e.g. glutathione synthetase, superoxide dismutases, catalases) and low-molecular substances (e.g. cysteine, glutathione, flavins and the vitamins C, E), and on the other hand specific repair systems which recognize DNA modifications and enzymatically remove the same from the DNA. The effectiveness of the defense mechanisms may, however, vary considerably from individual to individual and from cell type to cell type within an individual. Their efficiency is decisive for the tumor sensitivity of an individual to carcinogenic factors. The present invention especially deals with the determination of the activity of repair systmes (i.e. the repair capacity) as well as applications of such activity determinations.

B. Prior Art

For determining the repair capacity of human cells or tissues for various carcinogen-DNA modifications a multitude of methods are available. The most commonly used methods are listed below. In principle, they can be divided into two different types of method.

I. One type of method is based on the treatment of cells ex vivo with a certain carcinogen in vivo. What is then measured is the rate at which the carcinogen-generated DNA modifications are enzymatically removed from the DNA of the cells. For this purpose, the DNA of the cells is analyzed at various times after the carcinogen treatment for the remaining amount of corresponding DNA modifications. The data thus obtained are used for calculating the cellular repair capacity.

I.I. For determining the amount of defined DNA modifications, the DNA is isolated from the corresponding cell samples and analyzed for the content of DNA modifications. The quantification of the DNA modifications in the individual DNA samples can be effected:

a) In intact DNA molecules according to the Immuno-Slot-Blot method by using antibodies against defined DNA modifications (Nehls et al., 1984b).

b) Upon enzymatic hydrolysis of the DNA in the individual deoxyribonucleotides

by means of the competitive radioimmunoassy by using antibodies against defined DNA modifications (Nehls et al., 1984a),

by means of an electrochemical detector system upon separation of the deoxyribonucleoside mixture with a HPLC apparatus (Floyd et al., 1986),

by mass spectroscopy upon separation of the deoxyribonucleoside mixture by means of gas chromatography (Dizdaroglu et al., 1985).

I.II. On the other hand, methods have been developed, by which DNA modifications can directly be measured in the individual cells. These methods include

a) the Immunocytologic Assay (Nehls et al., 1997), and

b) the Comet Assay (Ostling and Johnson, 1984).

II. Another type of method consists in incubating protein extracts from cells and tissues with DNA molecules which either contain defined DNA modifications (synthetic DNA molecules) or have been treated with certain carcinogens. What is then determined is the rate at which the respective DNA modifications are removed from the DNA molecules. This can be practiced with the following methods:

II.I. With the “DNA Nicking Assay” (Castaing et al., 1993). This method is used to demonstrate the elimination of DNA modifications by enzymatic excision from the DNA. The excision of DNA modifications is effected either in one step by mostly specifically acting endonucleases or in two steps by DNA glycosylases, which recognize and eliminate certain modified bases, and AP endonucleases, which excise the remaining apurinic and/or apyrimidinic sites from the DNA. In both cases, DNA nicks occur at the sites of the DNA modifications, which nicks lead to the original DNA molecule being shortened. For this assay, synthetic, radioactively labeled DNA molecules of a certain length are chiefly used, which include a defined DNA modification at a predetermined position. The quantitative determination of the still intact DNA molecules and of the shortened DNA molecules is effected upon separating the DNA molecules of different lengths by denaturing polyacrylamide gel electrophoresis.

II.II. With the Filter-Binding Test (Nehls and Rajewsky, 1990). This method is based on the observation that DNA-antibody complexes can be immobilized on nitrocellulose filters, whereas protein-free DNA is not retained. The principle of the method consists in determining the amount of DNA molecules which are still cabable of binding antibodies. For this purpose, DNA which (ideally) contains one DNA modification per DNA molecule is incubated with protein extracts, mixed with a specifically binding antibody after various reaction times, and filtered through nitrocellulose filters. From the rate at which the amount of filter-bound DNA-antibody complexes decreases, the repair capacity of a cell type or tissue for defined DNA modifications, against which antibodies are available, can be determined.

II.III. Most of the DNA modifications are eliminated by excision repair. One exception are certain DNA modifications which are generated by alkylating carcinogens (e.g. O⁶-alkylguanine, O⁴-methylthymine). These alkylation products can be repaired by an enzyme (O⁶-alkylguanine-DNA-alkyltransferase); AT) which transmits the alkyl group from the DNA bases to itself and is then inactive. The important methods for the quantitative determination of this repair enzyme in cells and tissues are listed below.

a) Filter-binding test (see II.II.).

b) The principle of a frequently used test consists in determining the amount of a radioactively labeled O⁶-alkylguanine in a DNA treated with a [³H]-labeled alkylating agent prior to and at various times after the addition of cell and tissue extracts. Upon acid hydrolysis of the alkylated DNA, the purines released are separated by means of chromatography (HPLC, Sephadex G-10), and the radioactivity in the fraction containing O⁶-alkylguanine is determined (Foote et al., 1983).

c) Another frequently used method is based on the knowledge that the alkyl group is covalently transferred to a cysteine residue of the AT. Upon incubation of a [³H]-alkyl-DNA with protein extracts, the amount of alkyl groups transferred to the AT is determined by determining the radioactivity in the protein component of the extract. Alternatively, the amount of radioactivity remaining in the DNA is measured, and upon hydrolysis of the proteins the amount of [³H]-alkylcysteine molecules formed is determined (Pegg et al., 1983; Waldstein et al, 1982).

III. From WO 96/28571 a method for determining DNA damages is known, wherein DNA is fixed on a support by adsorption to a polycation. In this method, a composition containing a cell extract with repair activity and labeled nucleotides is allowed to act on the adsorbed DNA which has damages. The incorporation of the labeled nucleotides, which is effected in the case of repair, is then demonstrated.

IV. From the prior art, methods for covalently coupling biomolecules to solid supports are known, but not in connection with analyzing the repair of DNA damages.

From DE-A-43 41 524, for instance, there is known a method for immobilizing biomolecules and affinity ligands to polymeric supports by using a squaric acid derivative. DE-C-44 99 550 describes coupling reactions by using squaric acid derivatives and mentions the possibility of covalently linking biomolecules to a matrix. From DE-A-196 24 990 there is known a method for the chemically controlled modification of surfaces as well as of polymers carrying acyl and/or hydroxyl groups.

The above described methods for determining the repair capacity, which are known from the prior art, are in particular time-consuming, labor-intensive and expensive to execute. In some of the methods there is the problem of the loss of sample material during processing.

It is an object of the present invention to provide an improved method for analyzing the repair of DNA modifications, base mispairing as well as apurinic and apyrimidinic sites by DNA repair enzymes. In particular, the method should be simple, efficient and inexpensive to execute and avoid the above-mentioned disadvantages.

In accordance with the invention, this object is solved in that there is provided a method for analyzing the repair of DNA modifications and base mispairings as well as apurinic and apyrimidinic sites, comprising the following steps:

(a) providing single-stranded or double-stranded DNA molecules, which via a primary or secondary amino group incorporated in the DNA molecule at the 5′-end or at the 3′-end of the DNA or in the 2′-position of at least one deoxyribosyl residue were covalently coupled to a solid-phase matrix carrying primary or secondary amino groups by reaction with a reactive squaric acid derivative, and which have modifications and/or base mispairings and/or apurinic or apyrimidinic sites;

(b) bringing the DNA molecules in contact with a composition containing DNA repair enzymes;

(c) determining the elimination of the DNA modifications and/or base mispairings and/or apurinic or apyrimidinic sites.

In accordance with the invention, there are also provided test kits which comprise the components required for executing the method in accordance with the invention.

Preferred aspects of the invention can be taken from the following description, the embodiments as well as the attached claims.

### C. SUMMARY OF THE INVENTION

The principle underlying the invention consists in that repair enzymes are allowed to act on DNA molecules, which are covalently bound to a solid support and have a modification and/or a base mispairing and/or an apurinic or apyrimidinic site (damage), and the elimination of the modification and/or base mispairing and/or the apurinic or apyrimidinic site is observed. The covalent linkage of the DNA molecules to the support is effected by means of a reactive squaric acid derivative. An expedient method for the qualitative or quantitative determination of the elimination of the modification and/or base mispairing and/or the apurinic or apyrimidinic site employs specific antibodies or antibody fragments against the modification and/or base mispairing and/or the apurinic or apyrimidinic site (or in the case of an apurinic or apyrimidinic site also against a derivative of such site). The binding content of such specific antibodies is determined. When the repair is effected by excision, the qualitative or quantitative detection can also be effected by observation of the loss of a suitably incorporated label; the label (or a binding region for a label) has thus been incorporated in a DNA segment which upon excision is no longer connected with the support. The amount of label released and/or label that remains bound can be determined.

By means of the inventive method it is, for instance, possible to analyze the repair capacity of a composition containing repair enzymes for a predetermined DNA modification or base mispairing or apurinic or apyrimidinic site. On the other hand, however, DNA modifications or base mispairings or apurinic or apyrimidinic sites can be detected by using defined repair enzymes. Furthermore, the influence of agents on DNA can be tested by analyzing modifications caused by the influence of the agents (e.g. by specifically acting repair proteins) as well as the repair thereof. Statements can thus be made on the DNA-damaging potential of the agents. Finally, it is also possible to analyze the influence of reaction conditions and in particular of substances on the course of the repair.

The repair capacity generally designates an activity during the elimination of DNA modifications or base mispairings or apurinic or apyrimidinic sites. In particular, the repair capacity as it is understood here is a measure for the decrease in the content of DNA modifications or base mispairings or apurinic or apyrimidinic sites in a sample. Quantitative analyses will be explained in the examples, and the mathematical relations indicated there will generally be applicable in the respective type of analysis method.

### D. DETAILED REPRESENTATION OF PREFERRED EMBODIMENTS

Among other things, the invention thus relates to a method by means of which the capacity of cells or tissues for the enzymatic repair of defined modifications of the DNA structure (also referred to as DNA modifications, DNA damages or DNA adducts) and DNA mispairings can be determined quickly and precisely. Furthermore, when using defined DNA repair enzymes, the nature of the modifications of the DNA structure and of the base mispairings can be analyzed. Accordingly, the invention also provides a method for determining the repair capacity of modifications of the DNA structure, of base mispairings and apurinic or apyrimidinic sites by compositions (in particular solutions) containing DNA repair enzymes, which method can also be used for detecting modifications of the DNA structure, base mispairings and apurinic or apyrimidinic sites themselves, comprising the following steps:

(a) Single-stranded or double-stranded DNA molecules or DNA analogs, which were modified such that at the 5′-end or at the 3′-end of the DNA or in the 2′-position of at least one deoxyribosyl residue they carry a primary or secondary amino group inside the DNA molecule, and which may have modifications of the DNA structure and/or base mispairings and/or apurinic or apyrimidinic sites, are covalently coupled to the matrix by reaction with a squaric acid ester via a solid-phase matrix carrying primary or secondary amino groups;

(b) bringing the DNA molecules bound to the solid-phase matrix in contact with a solution containing repair enzymes;

(c) qualitative and/or quantitative determination of the repair of possible structural modifications and/or base mispairings and/or apurinic or apyrimidinic sites by means of the repair enzymes present in the solution.

In the case of the determination of a structural modification and/or base mispairing and/or apurinic or apyrimidinic site defined repair enzymes are used, which are capable of repairing a specific structural modification and/or base mispairing and/or apurinic or apyrimidinic site.

The repair capacity can vary considerably both from cell type to cell type inside an individual and also from individual to individual.

Accordingly, it is an important parameter for

the individual tumor susceptibility,

the sensitivity of tumor patients to a radiation therapy or genotoxic chemotherapy,

the resistance of tumor cells to a radiation therapy or genotoxic chemotherapy.

The individual tumor susceptibility is connected with the capacity of cells of an individual to repair DNA modifications and/or base mispairings and/or apurinic or apyrimidinic sites. By means of the inventive method, the repair status of an individual and thus the tumor susceptibility can be determined in that a composition suitably obtained from cells or tissue samples is allowed to act on a DNA which has modifications or base mispairings and/or apurinic or apyrimidinic sites, and in that the elimination thereof is demonstrated. When it is known that a certain carcinogen causes a certain type of modification and/or base mispairing and/or apurinic or apyrimidinic site, the repair status can be determined in this respect and hence the tumor susceptibility for the carcinogen can be established.

The determination of the individual radiation sensitivity is of great importance in so far as in a radiation therapy, e.g. of cancer diseases, about 30% of the patients require an inpatient treatment because of damages of healthy radiation-sensitive tissues adjacent the tumor, permanent damages being even caused in about 2%. On the other hand, it would sometimes be desirable for an optimum control of the tumor to employ radiation doses higher than usual. Upon collection of a sample of a healthy tissue, which during the therapy is exposed to radiation, or of a suitable surrogate tissue, a composition is prepared upon collection for the further analysis (a suspension or an extract), in order to determine the radiation sensitivity in accordance with the invention. Subsequently, the kinetics of the repair of DNA damages are analyzed. For this purpose, the aforementioned composition is allowed to act on DNA molecules in which before or after coupling one or more suitable modifications and/or base mispairings and/or apurinic or apyrimidinic sites have been incorporated. The radiation sensitivity can now be determined in that the decrease of the amount of DNA modifications or base mispairings or apurinic or apyrimidinic sites as a function of time, in particular the amount of modifications caused by reactive oxygen species, e.g. the amount of 8-oxoguanine, is measured and correlated with standard data. It is thus possible to individually determine the total radiation dose and the distribution of the single doses over time in the case of a fractionated irradiation. In accordance with a special embodiment, it is in addition provided to combine the data determined as described above, which represent the repair capacity of the cells with respect to DNA damages, with data describing the proliferation of the cells, in order to obtain an even more precise determination of the radiation dose. Correspondingly, the radiation sensitivity of tumor tissue can be determined in order to estimate the radiation dose required for a treatment.

Analogous to the determination of the sensitivity of tumor patients to a radiation therapy, the sensitivity to a genotoxic chemotherapy can also be determined. The repair capacity is determined in particular for one or more suitably chosen DNA modifications. When the mode of action of the chemotherapeutic agent is known, DNA modifications can be chosen on the basis of this mechanism. For instance, in connection with an alkylating agent the repair of corresponding alkylated bases can be analyzed. The same considerations apply to the determination of the resistance of tumor cells to a certain agent.

The basis of the method for analyzing the repair is the covalent linkage of modified DNA molecules or DNA molecules with base mispairings or apurinic or apyrimidinic sites to solid phases such as filters, gold, beads, microtitre plates or glass surfaces. Suitable supports are in particular chips (DNA-chip technology). The immobilized DNA is incubated with protein extracts from cells or tissues. The rate is measured at which defined DNA adducts or base mispairings or apurinic or apyrimidinic sites are removed by the repair proteins contained in the extracts.

As coupling agent a reactive squaric acid derivative is used, which is capable of reacting with amino groups. There is preferably used a squaric acid diester, in particular a squaric acid dialkyl ester, such as especially squaric acid diethyl ester, which can each link two primary or secondary amino groups with each other. Surprisingly, the squaric acid derivatives used in accordance with the invention only react with aliphatic amino groups, but not with the nitrogen atoms of the DNA bases. This is an inestimable advantage over other coupling agents, which react with the reactive groups of the DNA and can thus lead to undesired damages (e.g. dialdehydes). By introducing a primary or secondary amino group at the 5′-end or at the 3′-end or in the 2′-position of at least one deoxyribosyl residue inside the DNA molecules there was thus discovered a gentle, highly reproducible and inexpensive method for the regiospecific immobilization of single- and double-stranded oligonucleotides to solid phases which carry amino groups at their surface. The amino group should preferably be introduced at the 5′-end. The introduction of a primary amino group (NH₂ group) is particularly preferred. In the case of double-stranded DNA molecules preferably one strand is covalently linked to the support, e.g. via its 5′-end.

As solid-phase matrix, materials known per se may be used, for instance those consisting of cellulose, polystyrene, polypropylene, polycarbonate, a polyamide, glass or gold surfaces. The solid-phase matrix may be present in forms known per se, for instance in the form of a filter, in the form of microtitre plates, membranes, columns, beads, for example magnetic beads.

In accordance with the invention, the term DNA molecules comprises single-stranded and double-stranded molecules with any natural or synthetic sequence. Any number of bases in the DNA molecule may be chosen, if a sufficient number of bases is available for interaction with the repair enzyme. In some cases, oligonucleotides with few bases may already be sufficient. For some repair enzymes it turned out to be expedient to use oligonucleotides with three complete windings, the modification or mispairing or apurinic or apyrimidinic site being disposed in the middle winding. However, the length of the sequence and the arrangement of the site to be repaired can be varied by a person skilled in the art in routine experiments. The person skilled in the art can likewise analyze variations in the sequence. It turned out to be advantageous to use sequences which do not allow refolding. By variations of the sequence, the person skilled in the art can also analyze the influence of the environment on the repair of the site to be repaired.

As has already been mentioned, an amino group has been incorporated in the DNA molecules for coupling to the support.

Furthermore, modifications or base mispairings or apurinic or apyrimidinic sites have been incorporated, which can be repaired. Moreover, the term DNA molecules also includes DNA analogs.

As DNA analogs, DNA molecules modified at the phosphate-sugar backbone may, for instance, be used. Examples include molecules in which the phosphate groups have been replaced by phosphothiates (thiophosphates) or the phosphodiester bond has been replaced by a peptide bond. As DNA analogs molecules are considered in which some or all of the deoxyribonucleotides have been replaced by ribonucleotides.

In accordance with the invention, DNA repair enzymes are brought in contact with DNA molecules containing DNA modifications or base mispairings or apurinic or apyrimidinic sites which are believed to be capable of being repaired by the enzymes. Said alterations of the DNA may be caused directly or indirectly by carcinogenic factors (including radiation). The DNA modifications include in particular base modifications. Typically, these are adducts with reactive agents such as carcinogens. A DNA modification as understood here is, however, not restricted to a certain type of alteration of the DNA. In accordance with the invention, it is in particular also possible to specifically analyze synthesized DNA molecules, in particular oligonucleotides, with exactly defined modifications or base mispairings. Furthermore, modifications can also be generated by agents acting on DNA.

The covalent attachment of the DNA molecules to solid phases allows to quickly and efficiently perform all practical steps of an experiment in a single reaction vessel. Procedures such as the addition or the removal of enzymes, nucleic acids, antibodies or chromophoric substrates, the exchange of buffer solutions and washing operations, etc., only require largely automatable pipetting operations, i.e. there are no time- and labor-intensive working steps such as precipitation and repeated centrifugation of the DNA as well as the chromatographic or electrophoretic separation of the DNA molecules from other components. In addition, losses of DNA due to extensive processing of sample material can be avoided.

Another advantage of the invention consists in that the fixed DNA molecules are covalently attached to the support at a precisely defined point of the molecule, but otherwise are freely present in solution. DNA modifications, base mispairings and apurinic or apyrimidinic sites are thus freely accessible for repair enzymes.

Due to the use of various labeled substrates it is possible for the first time to analyze protein extracts from cells or tissues in the same reaction vessel for their repair capacity for various DNA modifications and/or DNA mispairings and/or apurinic or apyrimidinic sites. This is done in that various modified oligonucleotides are bound to the surface of the reaction vessels (or other supports), oligonucleotides with the same modification typically each including the same label or the same binding group for a label.

By means of the method, the repair status of a person can be determined quickly and precisely, and thus the susceptibility of this person with respect to carcinogenic factors in the environment or at the working place can reliably be estimated. The method can also be used for rapidly estimating the sensitivity of tumor patients (e.g. cells of the blood-forming system in the bone marrow, of the intestine and of the mucosa) to a radiotherapy or to genotoxic cytostatic agents. Finally, this method allows to quickly and precisely determine the repair capacity of tumor cells, which plays an important role in the resistance to DNA-reactive cytostatic agents.

The coupling method described here is basically suited for the gentle and regiospecific immobilization of oligonucleotides to solid phases.

A preferred embodiment of the method described here includes

the immobilization of selectively modified nucleic acids to solid phases, and

the use of squaric acid diethyl ester for covalently binding modified nucleic acid molecules to solid phases (such as filters, microtitre plates, membranes, glass surfaces, gold, beads and magnetic beads).

As coupling agent for covalently binding modified nucleic acid molecules, squaric acid diesters are recommended before all other agents.

Surprisingly, activated squaric acid derivatives, such as in particular squaric acid diesters, do not react with the amino groups of the purinic and pyrimidinic bases of the DNA. Rather, they selectively react with primary and secondary aliphatic amino groups. As compared to other coupling agents, this is a decisive advantage: An undesired damage or crosslinkage of the DNA molecules by the coupling substance does not occur.

By introducing amino groups e.g. at the 5′-end, DNA molecules can regiospecifically be linked to solid phases at whose surfaces amino groups are likewise present.

### E. Production of DNA Matrixes with Defined DNA Adducts by the Synthetic Incorporation of Certain Carcinogen-specific base Modifications as well as Production of DNA Molecules with Certain Base Mispairings or Apurinic or Apyrimidinic Sites

In the inventive method, DNA molecules with a fixed sequence and defined modifications or base mispairings or apurinic or apyrimidinic sites can be used advantageously. In the following, various approaches will be explained by way of example, which are, however, not meant to limit the invention. The required synthesis techniques are known to the person skilled in the art and can be taken from the literature.

I. Synthesis of single-stranded oligonucleotides of a defined sequence by means of the commonly used methods, which

a) have a certain base modification (e.g. 8-oxoguanine, O⁶-alkylguanine) at a predetermined point of the base sequence,

b) contain a NH₂ group at the 5′-terminus of the DNA molecules,

c) contain an OH group at the 3′-terminus.

I.I. Labeling the 3′-termini of the DNA molecules

Enzymatic prolongation of the 3′-termini by incorporation of e.g. fluorescence-labeled triphosphates with the terminal deoxynucleotide transferase (TdT). When various modified oligonucleotides are bound to the same support (simultaneous analysis of protein extracts for their repair capacity for various DNA modifications), various fluorescent dyes are used, oligonucleotides with the same modification each containing the same fluorescent dye. Alternatively, another label, or a group which can bind a label, can be incorporated. Moreover, it can easily be seen that the label need not be present at the 3′-end, but merely in such a position that upon excision it is no longer connected with the support.

I.II. Production of double-stranded (ds-) oligonucleotides:

a) Fusion of the modified oligonucleotides with the complementary oligonucleotides. (Preferably, the strand carrying the modification is thus covalently linked to the support.)

b) For analyses of the repair of DNA mispairings (mismatch repair) by protein extracts, unmodified or modified oligonucleotides are fused with complementary oligonucleotides which in a certain position of the base sequence or opposite the position of the DNA adduct contain different natural nucleotides.

I.III. Coupling of the ds-oligonucleotides at the 5′-end to a solid phase by means of a chemically stable or a cleavable linker. In general, it is possible in the present invention to provide linkers (spacers) which are linked to the surface of the solid support and at the end facing away from the surface carry amino groups via which coupling to DNA molecules is possible by means of squaric acid derivatives. A linker can likewise be connected with the DNA molecule, the linker in turn carrying an amino group which provides for squaric acid coupling. Inside the linker, a cleavable group may be provided, which allows the separation of the DNA molecule from the solid-phase matrix by means of suitable reagents for further analyses.

I.IV. When exclusively using uniformly modified dsoligonucleotides, the DNA molecules may for instance first be bound to a solid phase via the 5′-NH₂ termini, and at the 3′-end be subsequently enzymatically labeled

a) with a modified deoxynucleotide (e.g. biotinylated dUTP), to which a detectable label such as a fluorescent dye binds with high affinity (e.g. TRITC coupled to streptavidin), or

b) with a deoxynucleotide coupled to a fluorescent-dye (or radioactively labeled or labeled in some other way).

II. Production of DNA matrixes with specific DNA modifications by treatment of ds-oligonucleotides or linear plasmid DNA with carcinogenic factors (reactive chemical substances such as benzo(a)pyrenediolepoxide, methyl or ethyl nitrosourea, UV-light, ionizing radiation, hydrogen peroxide, methylene blue in conjunction with visible light).

II.I. Production of ds-oligonucleotides with NH₂ groups at the 5′-end and OH groups at the 3′-end of the molecules as described under item I. Treatment of the molecules with carcinogens. Binding the molecules via the NH₂ groups to a solid phase (see I.IV.) and enzymatic labeling of the bound molecules, for instance with a fluorescent dye.

II.II. Alternatively, the ds-oligonucleotides can first be bound to a solid phase. The treatment with a reactive carcinogen and the enzymatic labeling of the oligonucleotides will only be effected thereafter.

II.III. Introduction of NH₂ groups at the 5′-end of the plasmid DNA:

a) By means of the PCR method by using primers, one of which carries a NH₂ group at the 5′-end.

b) Cleavage of the plasmids by means of a restriction endonuclease, so that two new shorter DNA molecules are obtained, each with only one NH₂ group at one of the two 5′-termini/DNA molecule. Enzymatic incorporation of e.g. biotinylated dUTP at the 3′-ends of the plasmid molecules. Upon treatment with a carcinogen, binding the DNA matrixes to a solid phase is effected. In accordance with this embodiment, a detection reaction can be effected by means of a streptavidin-dye conjugate binding to biotin.

III. Production of DNA molecules with apurinic or apyrimidinic sites:

It is for instance possible to synthesize a DNA molecule into which a uridine monophosphate is incorporated, the base being separated exclusively enzymatically. There are also known enzymes which remove modified bases, an apurinic or apyrimidinic site being left.

### F. Practical Execution of the Repair Test

When performing repair tests, a composition assumed to contain repair enzymes is brought in contact with fixed DNA. The composition may be a cell extract or tissue extract. In this case, the inventive method allows to make statements on the repair activity of the cells or the tissue.

The detection of the elimination of certain DNA modifications or base mispairings or apurinic or apyrimidinic sites by enzymatic repair can be effected in two ways:

I. By using mono- or polyclonal antibodies specifically binding to certain DNA modifications (e.g. 8-oxoguanine, O⁶-alkylguanine, PAH-adducts of the DNA, pyrimidine dimers, etc.). Such antibodies are described in the literature. They can be produced by methods known to the person skilled in the art. There may also be used antibody fragments of suitable affinity.

This method is basically suited for detecting every DNA modification against which a suitable antibody is present.

Labeling the DNA molecules is not necessary with this procedure.

For a quantitative analysis of the repair capacity of a cell extract, the number of DNA adducts in the reaction preparation must be known; the number of DNA adducts per DNA molecule is irrelevant.

For this purpose, immobilized DNA molecules, which either contain a defined DNA modification (a defined DNA adduct) or have been treated with a certain carcinogen, are first of all incubated with a cell or tissue extract to be examined. Subsequently, an antibody is added, which specifically binds to the modification (primary antibody); for quantifying the amount of bound antibody a secondary antibody is then typically added, which is either labeled with a fluorescent dye (TRITC, FITC, fluorescein, etc.) or labeled in some other way, for instance radioactively, or to which an enzyme (e.g. phosphatase, catalase) is coupled, which with suitable substrates leads to a color reaction. Under constant conditions, the binding of the antibody molecules is directly proportional to the amount of remaining DNA adducts (Nehls and Rajewsky, 1990); i.e. the intensity of the dye is a measure for the amount of adduct.

Analogously, base mispairings or apurinic or apyrimidinic sites can also be analyzed with suitable antibodies. When analyzing the repair of apurinic or apyrimidinic sites it is also possible to derivatize these sites with suitable chemical agents, such as methoxyamines, hydrazine derivatives or substituted aromatic amines, and to use antibodies (or antibody fragments) against the derivatized sites for detection purposes. Expediently, derivatizing is effected upon action of the repair enzymes.

II. By utilizing the fact that DNA nicks occur when eliminating DNA modifications and base mispairings by excision repair.

This method is suited for detecting repair processes which lead to DNA nicks. Most of the known repair processes are effected according to this mechanism. One exception is the repair of e.g. O⁶-alkylguanine by the AT (see C.II.III.).

For this method, ds-oligonucleotides are preferably used.

For performing the method, it must be ensured

a) that only one DNA strand (possibly) contains a defined DNA adduct,

b) that this DNA strand has been immobilized, and

c) that this DNA strand has been labeled with respect to the DNA modification such that upon excision the label is no longer connected with the support. For instance, when the DNA strand is covalently linked to the support via the 5′-end, a label may be provided at the 3′-end. In this case, the label should generally be disposed in a position 3′ with respect to the point at which the DNA has been incised during repair. A structurally modified nucleotide, which is removed by excision, may also be labeled. In this case, a radiolabel is particularly useful. It is also possible to incorporate an additional label, which does not get lost during the repair (excision) and by means of which e.g. the amount of DNA immobilized can be analyzed in every stage of the method. For analyzing the elimination of DNA modifications, base mispairings or apurinic or apyrimidinic sites, as it has been described above, such possibly additionally provided label is, however, not suited.

For quantifying the repair efficiency of a cell extract, the number of DNA adducts per reaction preparation must be known.

Immobilized DNA molecules are incubated with a cell or tissue extract to be analyzed. When DNA adducts are removed by enzymatic excision, the covalently bound strands disintegrate in two fragments, of which the fragments with the label are no longer covalently bound to the solid phase. By heating the DNA molecules in a suitable buffer mixture, the hydrogen bridges between the two DNA strands are dissolved. The unbound fragments are thus separated from the complementary (non-labeled) counter-strands and can easily be removed e.g. by sucking them off. DNA molecules which have retained their DNA adducts still have the labels and can thus easily be quantified. Base mispairings as well as apurinic or apyrimidinic sites can be analyzed in the same way.

### G. BRIEF DESCRIPTION OF THE DRAWINGS

In the attached drawings,

FIG. 1 shows the kinetics of the removal of 8-oxoguanine from ds-oligonucleotides by cell extracts;

FIG. 2 shows the kinetics of the repair of O⁶-ethylguanine by cell extracts.

### H. EXAMPLES FOR APPLICATION

### Preparation of Modified Oligodeoxynucleotides

Three different oligodeoxynucleotides, each consisting of 34 nucleotides, were prepared, which contained a NH₂ group at the 5′-end. Except for position 16, the base sequence of the three oligonucleotides was identical and read as follows:

5′-GGC TTC ATC GTT ATT X ATG ACC TGG TGG ATA CCG-3′

where X in position 16 may be:

8-oxoguanine or O⁶-ethylguanine or guanine. As a base in position 16, the first oligonucleotide thus contained the oxidation product 8-oxoguanine, the second oligonucleotide contained the alkylation product O⁶-ethylguanine and the third oligonucleotide contained the natural base guanine. The third oligonucleotide served as control. In addition, the complementary DNA counter-strand was synthesized, which exclusively consisted of the four natural bases, and which protruded beyond the 3′-end of the modified oligonucleotides by two bases. Opposite X there was C. This provided for the enzymatic incorporation of biotinylated dUTP or fluorescence-labeled nucleotides at the 3′-end of the modified oligonucleotides or of the control oligonucleotide.

The oligonucleotides were prepared by the phosphoamidite method fully automatically. As usual, the synthesis was effected via the 3′-end at solid phases (CPG). Upon separation from the support material and cleavage of the protective groups (0.25 M 2-mercaptoethanol in concentrated ammonia, 55° C., 20 hrs.), the oligonucleotides were liberated from ammonia by gel filtration and subsequently purified by preparative polyacrylamide gel electrophoresis or HPLC. After another purification step, the oligonucleotides were lyophilized and stored at −20° C.

### Preparation of Double-stranded (ds) Substrates

Oligonucleotides dissolved in bidistilled water (100 pmol DNA molecules/ml) were mixed with 1.2 times the amount of the complementary DNA strand (120 pmol/ml). The samples were heated in a water bath for 5 min at 90° C.; the fusion of the single-stranded molecules to obtain double-stranded (ds) oligonucleotides was effected during the several hours' phase of cooling the water bath to room temperature.

### Preparation of Active Microtitre Plates

For the tests, microtitre plates (MPs) were used, whose wells contained flat bottoms and NH₂ groups at the surface (10 nmol NH₂ groups/well). Into the wells of the MPs, there were each pipetted 25 μl of a solution of squaric acid diethyl ester (0.1 mM) and triethylamine (0.01 mM) in methanol (>99%). The MPs were covered and incubated for 10 minutes at room temperature. Upon removal of the solution, the wells were washed with methanol and dried.

### Covalent Binding of ds-oligonucleotides to Activated MPs

For coupling the ds-oligonucleotides to the surface of the activated MP wells, 10 μl of the DNA solutions (50 pmol 8-oxoGua-ds-oligonucleotides/ml, 2.5 pmol O⁶-EtGua-ds-oligonucleotides/ml) in 50 mM aqueous sodium borate solution, pH 9.5, were each pipetted into the wells, and the MPs were covered. After 20 minutes at room temperature, the DNA solutions were removed and the wells were washed with bidistilled water.

The remaining reactive groups of the squaric acid were inactivated with 30 μl of an aqueous ethanolamine solution (100 mM, pH 8.5). After 10 minutes, the MP wells were washed with bidistilled water and dried.

### EXAMPLE A

### Removal of 8-oxodeoxyguanosine from ds-oligonucleotides

### Type of Repair

Most of the DNA modifications, postreplicative base mispairings and apurinic or apyrimidinic sites are removed from the DNA by excision repair (an enzymatic process). The DNA oxidation product 8-oxoguanine (8-OxoGua) is repaired according to the same mechanistic principle. In humans there are at least two different repair systems which eliminate this base modification from the DNA. In mice, only one of the two repair systems was detected.

Independent of the mechanism of the 8-OxoGua-excision, a gap having the size of a nucleotide is intermediately formed in the DNA.

### Practical Execution of the Test

For the test, MPs were used in whose wells with flat bottoms ds-oligonucleotides (30×10⁻¹⁵ mol dsDNA-molecules/well) were immobilized, which in position 16 contained the oxidation product 8-OxoGua and in positions 35 and 36 biotinylated uracil.

The cell extract to be analyzed was prepared from mouse myeloma cells of the cell line P3-X63-Ag. For this purpose, the cells were washed two times in ice-cold PBS and resuspended in buffer mixture A (50 mM Tris-HCl, pH 7.6, 1 mM EDTA, 1 mM DTT, 100 mM KCl, 0.1% BSA) in a concentration of 5×10⁷ cells/ml. The cells were disintegrated by sonication, and the solid constituents were removed by centrifugation (10,000 g, 4° C., 10 min). The clear supernatant was stored in portions at −80° C.

Into ten wells of an MP, 25 μl of a solution were pipetted, which contained the buffer mixture A and the protein extract of 6×10⁵ mouse myeloma cells (sample field).

Into four fields, 25 μl of the same buffer, but without protein extract, were pipetted (control field). The MP was incubated at 37° C.

After 5, 30, 60, 90 and 120 minutes, the reaction was stopped by adding 1 μl proteinase K (1 mg/ml) in two wells each per point in time. Into the wells of the control field, 1 μl proteinase K was likewise added after 120 minutes. The MPs were heated to 90° C. for 5 minutes and subsequently quickly cooled in an ice-water mixture. The solutions were removed from the wells, and the wells were washed three times with 30 μl of buffer B (50 mM Tris-HCl, pH 7.6, 1 mM EDTA, 0.1% BSA).

Upon adding 25 μl of a solution containing a streptavidin-Cy3 conjugate (2.5 μg/ml; obtained from the firm Sigma) in buffer B, the MP was incubated for 45 minutes at 37° C. Upon removing the solution, the wells of the MP were rinsed three times with buffer B, and finally 25 μl of the same buffer were added.

The intensity of the fluorescent dye in the individual wells was determined quantitatively by means of an image analyzer comprising a fluorescence microscope, a CCD camera and a computer-assisted analysis program. Alternatively, a sensitive UV-ELISA reading device can be used for measuring the fluorescence intensities.

The calculation of the samples was effected according to the formula

R=M(1−P/K₀)

wherein

R is the amount of repaired 8-OxoGuanine molecules in fmol (10¹⁵ mol),

M is the total amount of 8-OxoGuanine molecules in each MP well,

K₀ is the fluorescence intensity in the wells of the control field, and

P is the fluorescence intensity in the wells of the sample field.

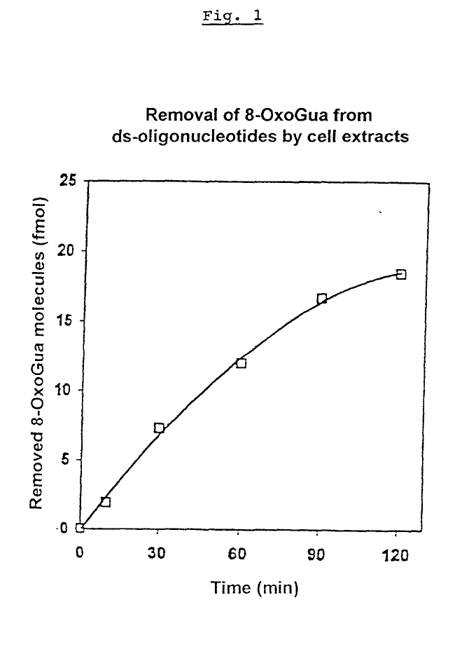

FIG. 1 represents the 8-OxoGuanine repair by a defined cell extract (extract of 6×10⁵ cells of a mouse myeloma cell line) as a function of the incubation time. From the initial slope of the curve it can be calculated how many 8-OxoGuanine molecules per hour are maximally removed from the oligonucleotides under standard conditions (total amount of 8-oxoguanine, 30 fmol; extract of 6×10⁵ cells). In the present case, 13.7 fmol/h were removed.

### EXAMPLE B

### Dealkylation of O⁶-ethylguanine in ds-oligonucleotides

### Type of Repair

The O⁶-ethylguanine (O⁶-EtGua) formed by alkylating carcinogens is repaired in one step by an O⁶-alkylguanine-DNA-alkyltransferase (AT). AT transfers an alkyl group from the O⁶-position of the guanine to a cysteine in the active center of the protein. By the transfer of the alkyl group, the AT is inactivated. Thus, each AT molecule can always repair only one O⁶-EtGua molecule. Accordingly, the repair is effected in a bimolecular reaction.

The repair can be analyzed by means of antibodies against O⁶-ethyldeoxyguanosine. Monoclonal or polyclonal antibodies may be used. Monoclonal antibodies can, for instance, be produced as follows:

First of all, the synthesis of O⁶-ethylriboguanosine and the coupling of the alkylation product to KLH (keyhole limpet haemocyanin) are effected as described by R. Muller and M. F. Rajewsky (Z. Naturforsch., 33c, 897-901, 1978). For producing antibodies, the antigen BALB/c is injected i.p. into mice five times over a period of three months. Spleen cells of the immunized mice were fusioned with myeloma cells (P3-X63-Ag8). The hybridomas thus obtained were sown onto microtitre plates containing peritoneal macrophages of BALB/c mice. Hybridoma cells producing antibodies against O⁶-ethyldeoxyguanosine were recloned and cultured. The antibodies were separated from other proteins in two purification steps (ammonium sulfate precipitation, 50% saturation, and ion exchange chromatography with DE-52 column material). The purified antibodies are concentrated and stored in portions at −80° C.

### Practical Execution of the Test

For the test, MPs were used in whose wells dsoligonucleotides (1.2×10⁻¹⁵ mol dsDNA molecules/well) were immobilized, which in position 16 contained an O⁶-EtGua. The 3′-ends were not filled in. In part of the wells, dsoligonucleotides were immobilized, which in position 16 merely contained a guanine (control field 2).

The cell extract to be analyzed was prepared by washing L929 mouse fibroblasts upon treatment with trypsin EDTA once in culture medium (DMEM, supplemented with 10% fetal calf serum) and twice in ice-cold PBS. The cells were then resuspended in extraction buffer (500 mM NaCl, 50 mM Tris-HCl, pH 7.8, 1 mM dithiothreitol, 1 mM EDTA and 5% glycerol) in a concentration of 6×10⁷ cells/ml and disintegrated by sonication. The insoluble constituents were removed by centrifugation (10.000 g, 4° C., 10 min) and the clear supernatant was stored in portions at −80° C.

Into 14 wells of an MP 50 μl of a solution were pipetted, which contained a reaction buffer (50 mM HEPES, pH 7.8, 1 mM DTT, 1 mM EDTA, 5% glycerol and 0.05% Triton X-100) and 3 μg protein extract from L929 mouse fibroblasts (sample field).

Into four wells, 25 μl of the reaction buffer without protein extract were pipetted (control field 1). Into four wells of control field 2, which contained unmodified dsoligonucleotides for determining the unspecific binding of the antibodies, reaction buffer and protein extract were pipetted.

After 5, 10, 15, 20, 30, 45, 60 minutes the reaction was stopped by adding 1 μl proteinase K (1 mg/ml) in two wells each per point in time. Into the well of control fields 1 and 2, 1 μl proteinase K was likewise added after 60 minutes.

After another 15 minutes at 37° C., the solutions were removed from the wells and the wells were washed three times with 30 μl buffer C (100 mM NaCl, 10 mM Tris-HCl, pH 7.5, 1 mM EDTA and 0.1% BSA).

Then, 15 μl of a solution containing 2 μg of an anti-(O⁶-EtGua) antibody in buffer C were pipetted into each of the wells. After 45 minutes at room temperature, the antibody solution was removed. Subsequently, the wells were washed three times with buffer C to remove unbound antibody molecules.

For detecting the specifically bound antibody molecules, 20 μl TRITC-labeled anti-(IgG) F(ab)₂ fragments in buffer C (5 μg/ml) were added into the wells. After 45 minutes at room temperature, the antibody solution was removed. Unbound antibody was removed by washing three times with 25 μ1 buffer C. Subsequently, 25 μl buffer C were pipetted into the wells. The determination of the fluorescence intensity in the individual wells was effected as before.

The calculation of the individual sample values was effected according to the formula

R=M(1−(P−K₂)/(K₁−K₂))

wherein

R is the amount of repaired O⁶-EtGua molecules in fmol (10⁻¹⁵ mol),

M is the total amount of O⁶-EtGua molecules in each well in fmol,

K₁ is the total fluorescence intensity in the wells without protein extract,

K₂ is the fluorescence intensity for the unspecific binding of antibodies,

p is the fluorescence intensity in the wells of the sample field.

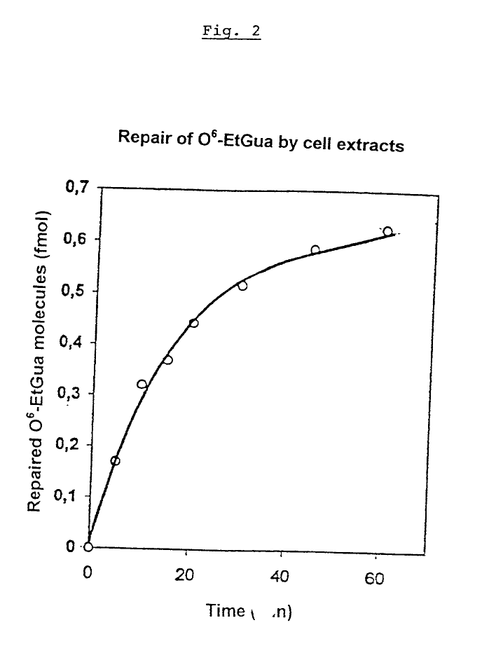

In FIG. 2, the repair of O⁶-EtGua by a defined cell extract (3 μg protein extract from L929 mouse fibroblasts) is represented as a function of time. Since the repair of O⁶-EtGua is effected in a bimolecular reaction (second-order reaction), the concentration of the AT molecules (AT) in the test preparation and the rate constant K of the repair reaction can be calculated according to the formula

K×t=1/(A₀−B₀)ln(B₀(A₀−P)/A₀(B₀−P))

wherein

the terms A₀, B₀ and P correspond to the initial concentration of the O⁶-EtGua molecules (A₀), of the AT molecules (B₀), and to the concentration of the dealkylated O⁶-EtGua molecules (P) at the time t of the repair reaction. For calculating AT and K a computer program was developed. Accordingly, the cell extract contained 0.7 fmol AT molecules, and K was 4×10⁷ l/mol×sec.

### Literature

Castaing, B., Geiger, A., Seliger, H., Nehls, P., Laval, J., Zelwer, C. and Boiteux, S. (1993) Nucleic Acids Res. 21, 2899-2905.

Dizdaroglu M. (1985) Biochemistry 24, 4476-4481.

Floyd, R. A., Watson, J. J., Harris, J. and Wong, P. K. (1986) Biochem. Biophys. Res. Commun. 137, 841-846.

Foote, R. S., Pal, B. C. and Mitra, S. (1983) Mutat. Res. 119, 221-228.

Nehls, P., Rajewsky, M. F., Spiess, E. and Werner, D. (1984a) EMBO J. 3, 327-332.

Nehls, P., Adamkiewicz, J. and Rajewsky, M. F. (1984b) J. Cancer Res. Clin. Oncol. 198, 23-29.

Nehls, P. and Rajewsky, M. F. (1990) Carcinogenesis 11, 81-87.

Nehls, P., Seiler, F., Rehn, B., Greferath, R. and Bruch, J. (1997) Environ. Health Perspect., 105 (Suppl. 5) 1291-1296.

Ostling, O. and Johanson, K. J. (1984) Biochem. Biophys. Res. Commun. 123, 291-298.

Pegg, A. E., Wiest, L., Foote, R. S., Mitra, S. and Perry, W. (1983) J. Biol. Chem. 258, 2327-2333.

Waldstein, E. A., Cao, E. -H. and Setlow, R. B. (1982) Anal. Biochem. 126, 268-272.

1. A method for analyzing the repair of DNA modifications and base mispairings as well as apurinic and apyrimidinic sites by DNA repair enzymes, comprising the following steps:
(a) providing single-stranded or double-stranded DNA molecules, which via a primary or secondary amino group incorporated in the DNA molecule at the 5′-end or at the 3′-end of the DNA or in the 2′-position of at least one deoxyribosyl residue were covalently coupled to a solid-phase matrix carrying primary or secondary amino groups by reaction with a reactive squaric acid derivative, and which have modifications and/or base mispairings and/or apurinic or apyrimidinic sites;
(b) bringing the DNA molecules in contact with a composition containing DNA repair enzymes;
(c) determining the elimination of the DNA modifications and/or base mispairings and/or apurinic or apyrimidinic sites.

2. The method as claimed in claim 1, wherein the reactive squaric acid derivative is a squaric acid diester.

3. The method of claim 2, wherein said squaric acid derivative is squaric acid diester.

4. The method as claimed in claim 1, wherein the elimination of the DNA modifications and/or base mispairings and/or apurinic or apyrimidinic sites from the DNA molecules coupled to the solid-phase matrix is determined by means of antibodies against the modifications and/or base mispairings and/or apurinic or apyrimidinic sites and/or derivatives of apurinic or apyrimidinic sites or corresponding antibody fragments.

5. The method as claimed in claim 1, wherein the elimination of the DNA modifications and/or base mispairings and/or apurinic or apyrimidinic sites from the DNA molecules coupled to the solid-phase matrix is determined by excision repair, in that the loss of a DNA segment is detected, which upon excision of the modifications and/or base mispairings and/or apurinic or apyrimidinic sites is no longer connected with the solid-phase matrix.

6. The method as claimed in claim 5, wherein the DNA segment lost by excision repair contains labels or groups binding labels.

7. The method as claimed in claim 6, where said labels are selected from the group consisting of chromophoric molecules, fluorescent molecules and radioactive atoms.

8. The method as claimed in claim 6, wherein the labels or the groups binding labels are incorporated upon coupling the DNA molecules to the solid-phase matrix.

9. The method as claimed in claim 5, wherein DNA molecules having various modifications or base mispairings or apurinic or apyrimidinic sites are each provided with various labels or groups binding labels, in order to provide for simultaneously analyzing the repair of various DNA modifications or base mispairings or apurinic or apyrimidinic sites.

10. The method as claimed in claim 1, wherein modifications are incorporated in the DNA molecules in that a modifying agent is allowed to act on the DNA molecules coupled to the solid-phase matrix.

11. The method as claimed in claim 1, wherein the repair capacity of the composition containing repair enzymes is determined for one or more DNA modifications or base mispairings or apurinic or apyrimidinic sites.

12. The method as claimed in claim 11, wherein the composition containing repair enzymes is recovered from cells or tissue samples, and the repair capacity is used to determine the individual tumor susceptibility, the individual radiation sensitivity or the individual sensitivity to a genotoxic chemotherapy or the resistance of tumor cells to radiation or chemotherapeutic agents.

13. A test kit for performing the method as claimed in claim 1, comprising:
(a) a solid-phase matrix carrying primary or secondary amino groups;
(b) DNA molecules which have a primary or secondary amino group incorporated in the DNA molecule at the 5′-end or at the 3′-end of the DNA or in the 2′-position of at least one deoxyribosyl residue;
(c) optionally antibodies or antibody fragments against a DNA modification or a base mispairing or an apurinic or apyrimidinic site or a derivative of an apurinic or apyrimidinic site;
(d) a reactive squaric acid derivative.

14. The test kit for performing the method as claimed in claim 1, comprising:
(a) single-stranded or double-stranded DNA molecules, which via a primary or secondary amino group incorporated in the DNA molecule at the 5′-end or at the 3′-end of the DNA or in the 2′-position of at least one deoxyribosyl residue were covalently coupled to a solid-phase matrix carrying primary or secondary amino groups by reaction with a reactive squaric acid derivative, which DNA molecules may have modifications and/or base mispairings and/or apurinic or apyrimidinic sites, and may carry a label or a group binding labels;
(b) selectively antibodies or antibody fragments, which are specifically directed against a DNA modification or a base mispairing or an apurinic or apyrimidinic site or a derivative of an apurinic or apyrimidinic site.
